(19) United States
(12) Patent Application Publication
(10) Pub. No.: US 2007/0077575 A1
(43) Pub. Date: Apr. 5, 2007

(54) USE OF RNA POLYMERASE AS AN INFORMATION-DEPENDENT MOLECULAR MOTOR

(76) Inventors: William T. McAllister, Perth Amboy, NJ (US); Richard Pomerantz, Brooklyn, NY (US); Michael Anikin, Brooklyn, NY (US)

Correspondence Address: HOFFMAN WARNICK & D'ALESSANDRO, LLC, 75 STATE STREET, 14TH FLOOR, ALBANY, NY, 12207, US

(21) Appl. No.: 11/469,957

(22) Filed: Sep. 5, 2006

Related U.S. Application Data
(63) Continuation of application No. PCT/US04/06896, filed on Mar. 5, 2004.

(51) Int. Cl. C12Q 1/68; G01N 33/53; C12N 9/00

(52) U.S. Cl. 435/6; 435/7.1; 435/183; 977/702

(57) ABSTRACT

Materials and methods are described in which the information dependence of RNA polymerase is employed to enable its use as a molecular motor adaptable for movement within DNA grid arrays and to actuate, move, position or alter cargo such as physical structures and normally inanimate substances and objects.

### BACKGROUND OF THE INVENTION

Nucleotide polymerase enzymes are ubiquitous in nature and used extensively in the biotechnology industry. RNA polymerases are employed in nucleic acid amplification reactions with reverse transcriptase and RNaseH to amplify an RNA target using a methodology known as nucleic acid sequence based amplification. They are also widely used to synthesize messenger RNA (mRNA) from a DNA template, a necessary step in protein production. DNA polymerases are used to catalyze the formation of complementary DNA in the presence of DNA templates.

Single and multi-subunit RNA polymerase enzymes exist in nature. The multi-subunit RNA polymerase enzymes are found in bacteria, archaea, and eukaryotes. The single subunit RNA polymerase enzymes are found in some bacteriophages, mitochondria, some eukaryotic organelles and may be encoded by some eukaryotic plasmids. Although they share no apparent sequence or structural homology, both types of enzymes carry out the basic steps of transcription in an identical manner. To initiate synthesis, the enzyme binds to a specific promoter sequence in the DNA template that lies upstream of the start site for transcription. The enzyme then separates (melts) the two strands of the template near the start signal to form a transcription “bubble”, and begins RNA synthesis using the coding strand of the downstream DNA as a template and a single ribonucleotide as a primer. During the early stages of transcription, contacts between the RNA polymerase and the upstream promoter sequences are maintained while the active site translocates (extends) downstream. This results in the formation of a short RNA-DNA hybrid and extension of the transcription bubble. During this early stage of transcription, the enzyme engages in multiple cycles of initiation in which short RNA products are synthesized and released without movement of the enzyme away from the promoter (abortive initiation). When the hybrid reaches a length of ˜8-9 base pairs (bp), the promoter sequence is released, the melted promoter region collapses, and the 5′ end of the nascent strand of RNA is displaced, resulting in a more stable elongation complex (EC).

Bacteriophage T7 RNA polymerase (T7 RNAP) has been used as a prototype in the study of nucleic acid synthesis. The structure of bacteriophage T7 RNAP in the EC has been elucidated. See Tahirov, et al., Nature 420 (6911): 43-50 (2002); Tahirov, et al., Acta Cryst. D59: 1 85-87 (2003). The transition from unstable initiation complex (IC) to stable EC is accompanied by conformation changes that results in a shell-like architecture. Downstream DNA is bound in a deep groove and enters through a wide passage to a cavity that contains an 8 bp RNA-DNA hybrid. The structure contains partly accessible channels and prominent pores for entry of the substrate and exit of the RNA product. A positive charge covering almost the entire interior of the molecule extends through the pores and channels to the external surface. This overall organization of T7 RNAP EC bears a significant resemblance to that of the multi-subunit RNAPs and the structural mechanism by which T7 RNAP achieves the EC configuration is similar to the steps observed in bacterial RNAPs. The reorganization of the enzyme as it makes its transition to EC appears to be a consistent theme among DNA-dependent RNA polymerases (RNAPs) in general. Tahirov, 2002, supra.

Once the EC is formed, RNA polymerase is able to transcribe great lengths of DNA (tens of thousands of bases) without dissociating from the template. Advancement of the complex at each polymerization step depends upon the availability of a ribonucleotide substrate that is complementary to the next base in the DNA template. If the required substrate is not present, the progress of the polymerase is halted. The halted transcription complexes are usually quite stabile, and transcription is resumed upon addition of the missing substrate. See Gopal, et al., J. Mol. Biol.: 411-31 (1999). Immobilization of transcription complexes on a solid surface permits repeated cycles of transcription to be carried out in the presence of limited mixtures of substrate.

Single molecule studies of the multi-subunit RNAP of E colihave shown that RNAP can exert considerable force as it moves along the DNA template. In such studies, the enzyme was immobilized on a solid surface. Movement of the DNA template through the immobilized complex (as a result of transcription) was monitored by tethering a reporter ligand to the downstream end of the template. See Davenport, et al., Science 287. 2497-2500 (2001); Harada et al., Nature 409:113-15 (2001); Mehta, et al., Science 283: 1689-95 (1999); Wang et al., Biophysical Journal 74: 1186-1202 (1998); Wang et al., Science 282: 902-07 (1998); Yin et al., Science 270: 1653-57 (1995).

Biological motors have been described that can exert linear or rotary forces, for example kinesin, myosin and F₁-ATPase. However, none of these may be precisely controlled, especially in an information-dependent manner.

While the multi-subunit E. coli RNAP has been shown to be able to exert force, its use as a biological motor apparently has not been suggested or attempted, perhaps due to the following difficulties. Because the endogenous multi-subunit RNAP is required for cell growth, any modifications deemed desirable may prove lethal or make purification of a homogeneous RNAP population from the bacterial cell culture difficult or unwieldy. In addition, experiments with the multi-subunit E. coli RNAP demonstrate that when halted at certain sites, the enzyme can enter into an irreversibly arrested state (“dead end” complex) or may slide back along the DNA template and cleave the nascent RNA before recovering (“backtracking”). Furthermore, single molecule studies of E. coli RNAP have indicated that movement of the enzyme along the template is not regular, and that progressive transcript elongation is often interrupted by pauses of an apparently random nature. These stochastic interruptions in enzyme activity may be related to the backtracking and arrest phenomena noted above. The formation of dead ends or backtracked complexes has also been observed with other multi-subunit RNAPs. These problems make use of the multi-subunit nucleotide polymerases as molecular motors, i.e., motors capable of actuating the movement of structures or molecules, theoretically possible, but more problematic and impractical from an industrial standpoint than the use of the single subunit nucleotide polymerases.

Thus, it would be advantageous to provide a molecular motor capable of exerting forces that can be precisely controlled in an information dependent manner and easily manipulated. Such motors would be capable of capturing and moving (i.e., actuating) biological molecules or inorganic molecules or particles with accuracy and stability, and could be usefully applied in various aspects of nanotechnology.

### SUMMARY OF THE INVENTION

In one aspect, the invention is a molecular motor for actuating macromolecules or molecular devices in the manner of “cargo”. The motor comprises a nucleotide polymerase (NP) enzyme having a high affinity-binding domain capable of allowing the enzyme to bind to a ligand or ligands (which could be the “cargo” for example, or a structural element) and/or to a solid surface and having the ability to exert movement and force in an information dependent manner. By “information dependent” we mean that the movement of the RNAP and the bound macromolecule or particle or molecular device depends upon the availability of the next ribonucleotide substrate(s) to be incorporated by the polymerase motor as directed by the sequence of the template strand. Because advancement of the polymerase depends upon the availability of a ribonucleotide that is complementary to the DNA template, polymerase movement can be controlled in a sequence-specific, information-dependent manner by providing or withholding appropriate substrates during each elongation cycle.

By “high-affinity binding domain” we mean an amino acid, polypeptide or protein sequence that is fused to the RNAP and has or confers the ability to attach the NP to another substrate, either a solid support or particle or another sequence or molecule. In some cases, the binding may be reversible. Such a sequence also has to be able to bind tightly enough such that it is released only upon addition of a releasing agent or a modification in reaction conditions. Sequences comprising binding domains exhibiting affinities in the range of nM to pM Kd are exemplary of those that may be employed. Many high-affinity binding domains are known in the art. Exemplary are the yeast GAL4 binding protein, the zinc finger domains of DNA-binding proteins such as Zif268, the heavy metal binding domain of metallothionein, the DNA-binding domain of transcription factor Sp1 ³⁰, or the streptavidin binding domain of the streptavidin protein. Also, DNA or RNA aptamers may be employed as adapters to provide high-affinity, stereo-specific binding domains. For example, an aptamer constructed to include a defined recognition sequence such as the Zif268 binding motif may be attached to the NP enzyme:Zif268 fusion protein. The RNAP may also be modified in vivo to a biotinylated form, which has high affinity to streptavidin or to streptavidin-conjugated molecules. More than one binding domain may be attached to the NP.

The high-affinity binding domain can be reversibly or irreversibly bound by attachment or fusion to the NP at any site along its length as long as the functional ability of the NP or the DNA template is not disrupted or deleteriously affected. Preferably, the binding domain is bound at or near the N-terminus of the NP and in an irreversible manner.

The high-affinity binding domain also must be able to bind another entity, structure, substance or device, (the “cargo”). The cargo may be virtually anything: another solid support or bead, a biological small molecule or macromolecule, a peptide ligand, a polypeptide sequence, a protein or a portion of a protein, a DNA or RNA sequence, a typically inanimate substance or object, including inorganic objects or structures which may have useful properties such as semi-conducting materials, heavy metals, magnetic particles, or materials which exhibit desirable optical properties, i.e., anything that has the ability to bind to the high-affinity binding domain and be actuated by the information-dependent movement of the modified NP enzyme can be employed as the cargo.

As disadvantages in the use of multi-subunit polymerases can be overcome by the use of single subunit nucleotide polymerases, the latter are preferred for use as motors. While any single subunit nucleotide polymerase may be employed as a molecular motor, particularly preferred are the RNAPs encoded by bacteriophages T7, T3, SP6 and K11. Bacteriophage RNAPs are structurally simple, single subunit RNAPs that are easily manipulated. Manipulation of the gene encoding the enzyme allows addition of auxiliary domains conferring novel binding capacities. Because phage and other single subunit RNAPs are not required for cell growth, the modified gene may be expressed in bacterial cells without affecting the viability of the host. In addition, phage RNAPs, T7, T3, SP6 and K11 having distinct promoter specificities are readily available; this permits use of multiple RNAP motors each of which may be directed to a unique position on the template and separately controlled. In the examples, T7 RNAP is used because it the most well studied and understood nucleotide polymerase enzyme. However, any nucleotide polymerase enzyme (including DNA polymerases and reverse transcriptase) may be employed as a molecular motor if they contain or are modified to contain a high-affinity binding domain in such a manner that the functional ability or activity or the enzyme is not disrupted or deleteriously affected.

Because single subunit nucleotide polymerases appear to lack the dead ending and backtracking proclivities of the multi-subunit nucleotide polymerases, they are preferred. See, He et al., Protein Expression & Purification 9: 142-51 (1997). Both single subunit and multi-subunit nucleotide polymerases are readily and publicly available through a variety of sources.

Incorporated into the nucleotide polymerase is a high-affinity binding domain, which allows the enzyme to bind to a solid surface or to a ligand (or to both). By “bind to” we mean form a high affinity attachment with. Binding to would thus also include a covalent attachment. We use the terms “link to” and “fuse to” or “fuse with” in the same manner and with the same meaning and intent as “bind to”. The binding domain is fused to the enzyme at a position where it does not affect function activity, i.e., where it does not affect the enzyme's ability to synthesize RNA. For example, the binding domain may be fused to T7 RNAP at or near its N-terminus. In the following description and examples, different high-affinity binding domains are attached to T7 RNAP as an example of how the invention can be carried out. The skilled artisan should thus be able to readily design and test additional NPs modified with high-affinity binding domains. Also exemplified and described are some exemplary types of biological macromolecules that can be actuated, or moved, by the molecular motors of the invention.

In another aspect, the invention comprises a method of making a molecular motor. In the first step of the method, a NP enzyme is fused to a high-affinity binding domain capable of binding to a surface support or a ligand. The enzyme may be reversibly or irreversibly attached to this binding domain. The enzyme may be a single subunit or a multi-subunit nucleotide polymerase enzyme. The high-affinity binding domain can be a DNA sequence, a RNA sequence or an amino acid sequence. Exemplary DNA and RNA sequences include DNA or RNA aptamers. Exemplary amino acid sequences include the yeast GAL4 DNA binding polypeptide sequence, the Zif268 zinc-finger DNA-binding polypeptide sequence, a streptavidin-binding polypeptide sequence, a metallotheionein binding polypeptide sequence, a sequence-specfic DNA binding polypeptide from the transcription factor Sp1 ³⁰ and a 6 to 15 residue histidine sequence. In fact, any peptide sequence may be employed as long as it has a length and character so as to be able to form a strong bond with the cargo. The bonds formed may each be reversible or only the bond between the polymerase and the cargo may be reversible. The high-affinity binding domain can be bound, linked or fused to the NP anywhere along its length, so long as its enzymatic activity is retained. Preferably the high-affinity binding domain will be bound, linked or fused to the NP at or near its N-terminus. Other attributes of the high-affinity binding domain may be readily determined by the skilled artisan and will depend on the desired substance, ligand, cargo to be actuated or moved by the motor.

In another aspect, the invention comprises a method for moving substances or actuating cargo in an information-dependent manner. In the steps of this method, a solution containing a nucleotide triphosphate (NTP) substrate or a mixture of nucleotide triphosphate substrates is combined with a starting solution containing (a) a DNA template having a promoter sequence containing a polymerase binding site and a start site of transcription and (b) a nucleotide polymerase molecular motor of the invention. The nucleotide triphosphate substrates comprise GTP, CTP, UTP and ATP. NTPs complementary to the nucleic acids of the DNA template are added in a combination that allows the formation of a stable EC “start-up complex”. At least 14 nucleotides must be transcribed in order for the start-up complex to form. The three components may be combined simultaneously during the formation of the start-up complex or in an ordered manner. The combination or mixture is incubated under suitable transcription conditions to allow the formation of the start-up complex, and then washed to remove unincorporated substrate. Controlled movement of the motor is accomplished during subsequent cycles of substrate addition, incubation, and washing steps using substrates complementary to the subsequent (downstream) regions of the DNA template. Specific activity and nuclease activity can be tested using known methods and standard conditions.

In another aspect, the invention comprises an array of molecular motors composed of a plurality of identical phage nucleotide polymerases. Each of these polymerases may be fused to a different high-affinity binding domain and each may be arranged in a linear manner to form the array. In addition or alternatively, the array may be composed of a plurality of different phage nucleotide polymerases, resulting in the construction of an linear array of RNAP motors each with an unique promoter specificity. A plurality of such linear arrays may be arranged and positioned in a two dimensional grid.

### DESCRIPTION OF THE DRAWINGS

FIG. 1 illustrates the sequence-dependent, controlled movement of His₆ -tagged T7 RNAP as described in detail in Example 1.

FIG. 2 illustrates the loading and release of bound ligand during controlled walking of T7 RNAP as described in detail in Example 2.

FIG. 3 illustrates a simple DNA device, the construction and use of which is disclosed in Example 3.

FIG. 4 illustrates the rotary movement of polymerase enzyme along the DNA template during transcription as discussed in Example 4.

FIG. 5 illustrates the results of the experiment measuring the rotation of a magnetic bead by immobilized T7 RNAP as described in detail in Example 4.

FIG. 6 illustrates the construction of a parallel array of multiple RNAP motors on DNA bridges as discussed in detail in Example 5.

FIG. 7 illustrates the construction of two dimensional grids of DNA containing promoters for RNAP motors as discussed in detail in Example 6.

### DETAILED DESCRIPTION

During each cycle of nucleotide incorporation, the nucleotide polymerase enzyme advances 0.34 nm along the DNA template and exerts linear forces up to 30 pN. Since the progress of the polymerase depends upon the availability of the next ribonucleotide substrate(s) to be incorporated as directed by the sequence of the template strand, its movement can be restricted by providing or withholding appropriate substrates. While other biological motors can generate similar forces, none of them are controllable with the level of precision or in an information-dependent manner. In this manner, the nucleotide polymerase transcription complex may be stepped through multiple cycles of as few as one nucleotide or as great as hundreds or thousands of nucleotides per cycle.

In order to harness a nucleotide polymerase to accomplish useful work, the enzyme needs to be attached to another structure. In preliminary studies demonstrating the utility of employing RNAP as a molecular motor, we modified the single subunit RNAP encoded by bacteriophage T7 by N-terminally fusing to it a high-affinity binding domain to allow it to bind to solid surfaces and to other DNA molecules. We then demonstrated changes in simple DNA structures in a controlled manner. These studies are described in Examples 1 and 2 below.

Various high-affinity binding domains were employed. First, the enzyme was modified to include a hexahistidine (His₆) tag and bound via the tag to Ni⁺⁺-agarose beads or columns. Next, the His₆ tagged enzyme was modified to include a 38 amino acid SBP peptide tag that has a high affinity for streptavidin. The SPB peptide is compatible with a variety of streptavidin-conjugated fluorescent and enzymatic reporter systems and its binding to a ligand is readily reversible by the addition of biotin. In example 2, we use a strepatavidin-conjugated ³²P-labeled DNA fragment as the ligand, or “cargo”, and demonstrate loading, movement and release of the cargo during the stepwise movement of T7 RNAP along the DNA template. Although these studies used DNA as the biological macromolecule that is actuated by the NP molecular motor, this technology is readily applicable to the movement of other molecules or structures. For example, NP can be linked to ligand-specific aptamers of RNA or of DNA; it can be fused directly to ligand-specific peptide domains from other proteins such as streptavidin binding protein or metallothionein. In these ways, NP can be use to carry a variety of biological, organic and/or inorganic cargo.

In the crystal structure of T7 RNAP, the N terminus is solvent exposed and projects away from the surface of the enzyme. Sousa, et al., Nature 364: 593-99 (1993); Jeruzalmi and Steitz, EMBOJ. 17: 4101-13 (1998); Cheetham et al., Nature 399: 80-83 (1999); Tahirov 2002, supra; Yin et al., Science 298: 1387-95 (2002). Fusion of other peptides of protein domains to this region appears to have little effect on enzyme activity. He, supra.; Benton, et al., Mol. Cell. Biol. 10: 353-60 (1990); Rodriguez, et al., J. Virol. 64: 4851-57 (1990).

In the examples, we successfully fused three different biological macromolecules to RNAP to the N terminal region and moved them along a template as “cargo”. One was a sequence-specific GAL4 binding domain, another was a His₆ peptide, and the third was a SBP-tagged peptide. Due to the availability of a wide range of streptavidin-conjugated reporter systems, the SBP-tagged peptide modification is particularly useful for monitoring RNAP-ligand interactions. However, other binding motifs can be employed as the biological macromolecule, particularly sequence-specific DNA binding motifs such as tandem Zif268 three finger peptides. Such peptides may be incorporated using a flexible 11 amino acid linker sequence as described in Kim and Pabo, Proc. Nat. Acad. Sci. 94: 2812-17 (1998). This results in a six zinc finger fusion protein with extremely tight DNA binding.

Additional biological macromolecules comprise fusion proteins composed of other DNA binding domains from transcription factor Sp1 and the heavy metal binding domain of metallothionein. Methods and materials that can be used in the construction of these fusion proteins are disclosed in Kadanage, et al, Cell 52: 4851-57 (1990) and Sano et al., Proc. Nat. Acac. Sci. 89: 1534-38 (2002).

Aptamers of DNA or RNA are additional high-affinity binding domains that can be used as “adapter” or “linker” molecules. Apatamers are small nucleic acids selected from random libraries that are able to bind to other molecules with high affinity and specificity because of their ability to fold into unique structures. See Ellington and Szostak, Nature 346: 818-828 (1990); Tuerk and Gold, Science 249: 505-10 (1990). Tight, i.e., high affinity, binding of aptamers, in the range of nM to pM K_{d}, has been observed with organic molecules, carbohydrates, amino acids and peptides. See Gold et al., Ann. Rev. Biochem. 64: 736-97 (1995); Osborne and Ellington, Chem. Rev. 97:349-70 (1997). In a related manner, evolved peptides that have high affinity for a variety of ligands may also be derived by selection methods such as expressed phage peptide screening..

Because aptamers are typically produced by repeated cycles of selection and nucleic aid amplification, the most straightforward way to link an aptamer with the desired specificity to a RNAP motor is to incorporate a defined recognition sequence into the amplification primer. The Zif268 binding sequence could be used for this purpose. The recognition sequence would provide a “handle” for subsequent binding by a Zif268:T7RNAP fusion protein. Alternatively for RNA aptamers, the primer could include a specific single stranded region complementary to a DNA oligomer that contains the Zif268 recognition sequence; hybridization of the complementary regions of the aptamer and the DNA oligomer would permit the fusion protein to capture the aptamer.

Because of its ability to be precisely controlled by virtue of its dependence on the presence or absence of ribonucleotides, RNAP provides unique capabilities in its potential applications as a molecular motor. Coupling of RNAP to other materials finds utility in nanorobotics, the positioning of ligands or altering of structures with subnanometer precision, and in the assembly and movement of complex structures. For example, using existing technology, an array of RNAP motors could be assembled on a DNA grid immobilized on a solid surface. The movement of each RNAP in the array would depend upon the sequence of the DNA template to which it is bound and could be independently controlled.

The following examples employ bacteriophage T7 RNAP as a prototype RNAP molecular motor because T7 RNAP is the prototype of a class of single subunit enzymes that also includes RNAPs encoded by bacteriophages T3, SP6, K11 and others. Although similar in structure and function, each phage RNAP is specific for its own promoter sequence. We have shown that the basis for this specificity involves a DNA recognition loop that projects into the binding cleft of the RNAP and we have engineered mutant RNAPs with novel specificities. Joho, et al.,J. Mo. Biol. 215: 31-39 (1990); Klement et al.,J. Mol. Biol. 215: 21-29 (1990); Raskin et al., J. Mol. Biol. 228: 506-15 (1992); Raskin et al, Proc. Nat. Acad. Sci. 90: 3147-51 (1993); Rong et al., Proc. Nat. Acad. Sci. 95: 515-519 (1998). This feature of the phage RNAP system allows the construction of RNAP motors with unique promoter specificities, each of which may be fused to a different ligand-binding domain. Accordingly, this invention should not be limited to bacteriophage T7; RNAPs from any other bacteriophage could have been used. Engineered mutants such as those disclosed in the articles already cited can be used and are included in the scope of this disclosure. Modified bacteriophage RNAP from other bacteriophages, such as T3 bacteriophage can be used and are included in the scope of this disclosure. The nucleotide sequence of T3, plasmids for its production, transcription vectors carrying its promoter and promoter cassettes containing T3 and other phage RNAP promoters are described in U.S. Pat. No. 5,017,488 issued May 21, 1991; U.S. Pat. No. 5,037,745 issued Aug. 6, 1991; and U.S. Pat. No. 5,102,802 issued Apr. 7, 1992, incorporated by reference here. In addition, any single subunit RNAP, such as mitochondrial or organellar single subunit RNAPs and plasmid RNAPs, from a variety of non-bacteriophage sources can be equally employed. The flexibility inherent in the single subunit RNAPs affords great potential in designing arrays of RNAP motors for specific applications.

The following examples illustrate and present preferred embodiments of the intention. They are not to be construed as a limitation on the scope of the invention, as the skilled artisan will be able without undue experimentation to modify or make variants of the invention.

Materials and methods employed in this invention are described in the articles cited herein, each of which is incorporated by reference here for the substance of its disclosure, and in the well known texts used in the field such as Maniatis, Fritsch and Sambrook, Molecular Cloning: A LaboratoryManual, 2d Edition, Cold Spring Harbor Press, Cold Spring Harbor, N.Y., 1989; and Grossman and Moldave, Eds., Methods in Enzymology, Academic Press, New York, 1979.

### EXAMPLE 1

Immobilization and Controlled Movement of T7 RNAP

For many applications in which RNAP can be used as a molecular motor, it is necessary to attach the enzyme to a solid surface. One way in which this may be accomplished is to modify the enzyme to include a hexahistidine, His₆, tag. The tag allows the enzyme to bind tightly to Ni⁺⁺ agarose beads or columns, without affecting enzyme performance. See for example, Van Dyke, et al., Gene 111: 99-104 (1992). The methodology for modifying T7, T3 and SP6 phage RNAP to fuse histidine residues to the amino terminus, the oligonucleotides and other materials used in the modification, and the plasmid vectors containing the His-tagged RNAP (plasmids pBH116, pBH117, pBH161, pDL19, pDL21, pBH118, pBH176 and pDL18) are disclosed in detail in He, et al., Protein Expression and Purification 9: 142-151 (1997), specifically incorporated by reference here. The number of histidine residues added to the amino terminus is not critical, between 6 and 12 are preferred. In addition, the His leader sequence can also include a thrombin cleavage site. These modifications do not affect the properties or performance of the enzyme and other modifications may be made as long as the modifications do not affect the properties or performance of the enzyme.

The His-tagged RNAP is then advanced stepwise along a DNA template in a controlled, sequence-dependent manner as follows. His-tagged T7 RNAP was first incubated with a DNA template in the presence of a limited mixture of ribonucleotide substrates complementary to those of the DNA template beginning with the start of the promoter sequence and comprising a sufficient number and type to cause the formation of a stable EC. In our hands at least 14 nucleotides composed of those sequentially downstream of the start of the promoter sequence were necessary. This resulted in the formation of a halted, EC start-up complex. The ECs were then adsorbed to Ni⁺⁺ beads and washed to remove unincorporated substrates. Advancement of the RNAP along the DNA template requires the presence of ribonucleotides complementary to those specified by the template. The ECs in their halted state, the start-up complex, are quite stable and may be sequentially moved along the template during multiple cycles of washing and elongation. Elongation is strictly dependent upon the presence of the suitable substrate and may be controlled in increments of as little as 1 nucleotide, i.e., 0.34 nm advancement along the template. Furthermore, extension is nearly quantitative (>95%) at each step.

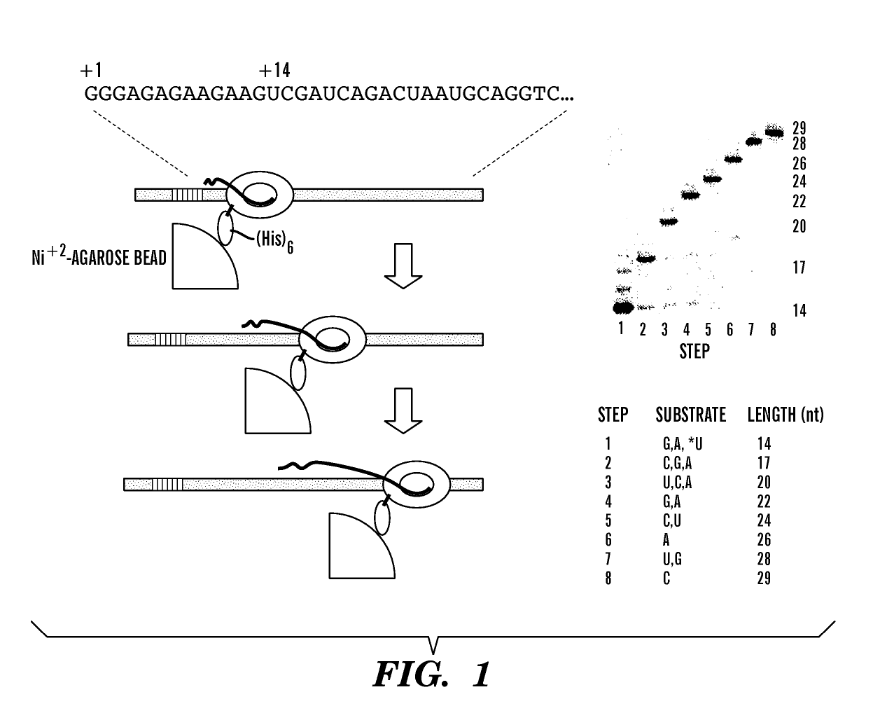

As illustrated in FIG. 1, His₆-tagged T7 RNAP, immobilized at its amino terminus on Ni⁺⁺ agarose beads was incubated with a template that directed transcription with the sequence indicated beginning at the start site (+1) in the promoter. In step 1, RNA synthesis was initiated by the addition of GTP, ATP and α-³²-UTP (G, A and *U respectively), which advanced the polymerase to nucleotide (nt) position +14 and resulted in the formation of a stable EC start-up complex. The beads were washed with buffer and then incubated with CTP (hereinafter C), G and A, which advanced the polymerase to position +17 (step 2). Successive cycles of transcription were carried out in the presence of the substrates indicated in the lower right of FIG. 1. After washing, in step 3, the addition of U, C and A advanced the polymerase to position +20. Washing again followed by the addition of G and A moved the polymerase to position 22 (step 4). Eight steps were carried out along a template length of 19 nucleotides. After each cycle of washing and transcription, a sample was removed and the RNA products analyzed by electrophoresis in 20% polyacrylamide gels in the presence of 0.1% SDS. A composite SDS-page gel is illustrated in the upper right of FIG. 1. Reaction and incubation conditions, volumes, amounts, buffers and enzyme preparations were as disclosed in Temiakov, et al., Protein:DNA Interactions: A Practical Approach(Travers, A A & Buckle, M, eds.), pp 351-64, Oxford University Press, Oxford, 2000.

This example demonstrates that T7 RNAP immobilized on Ni⁺⁺ beads can be “walked” along the DNA template in steps as large as 14 nt or as small as 1 nt during repeated cycles of washing and polymerization. The efficiency of extension at each step was extremely high (>95%), indicating that most of the transcription complexes in the population were extended during each cycle.

### EXAMPLE 2

Capture and Movement of Bound Ligand

In order to harness RNAP to do work, it is necessary to attach the enzyme to other structures or ligands. To demonstrate the ability of RNAP to bind another object during transcription, we modified His₆-tagged T7 RNAP to include an additional 38 amino acid peptide (SBP-tag) that has a high affinity for streptavidin (K_{D}=2.5 nM). The methods and materials employed in this modification are disclosed in Keefe, et al., Protein Expr. Purif 23: 440-46 (2001). The SBP-tag is compatible with a wide variety of streptavidin-conjugated fluorescent and enzymatic reporter systems and its binding to ligand is readily reversible by the addition of biotin. In this experiment, we used a biotin-conjugated ³²P-labeled DNA fragment as the reporter ligand.

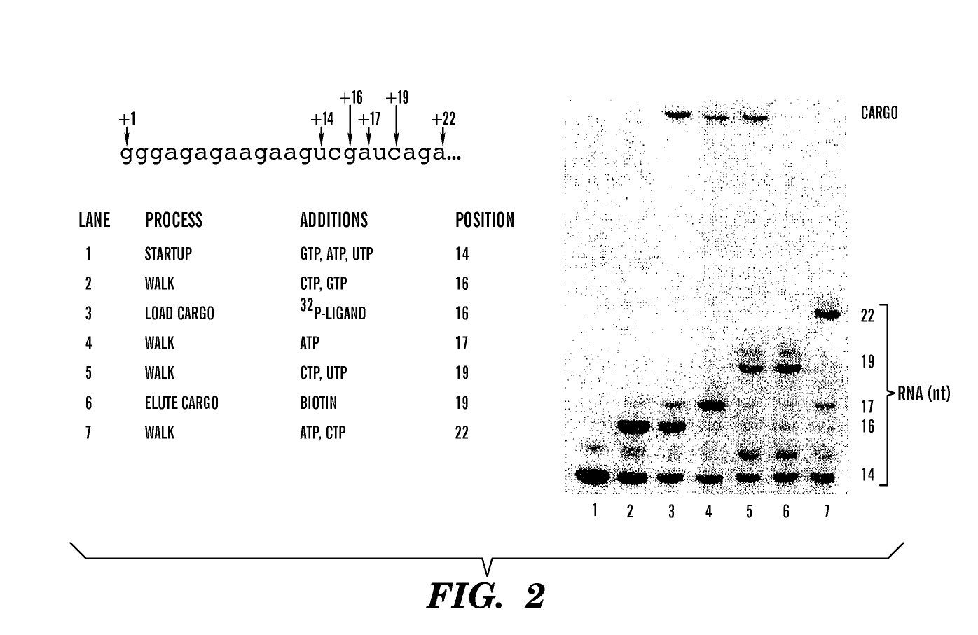

First, start up complexes of SBP-tagged RNAP were formed by incubation with a template that directs synthesis of an RNA with the sequence indicated in FIG. 2 in the presence of G, A and U, and the complexes were immobilized on Ni⁺⁺ agarose beads (Lane 1). This advanced the RNAP to nt position +14 and resulted in the formation of the stable EC start-up complex. After washing, the complexes were then “walked” 2 nt by the addition of C and G (Lane 2) to nt position 16 and washed again. Then the complexes were mixed with a 48 bp ³²P-labeled biotinylated DNA fragment that had been conjugated to streptavidin and washed (cargo; Lane 3). The cargo was then walked through two cycles, first by the addition of A, then by the addition of C and U (with washing steps intervening), to position +19 (Lanes 4 and 5). At position +19 the cargo was eluted by the addition of biotin (Lane 6) and the complexes were then advanced again by the addition of A and C (Lane 7). As in Example 1, after each cycle of washing and transcription, a sample was removed and analyzed by electrophoresis in 20% polyacrylamide gels in the presence of 0.1% SDS. A composite SDS-page gel is illustrated on the right side of FIG. 2.

Employing the same materials and methods referenced above, we fused a number of sequence-specific DNA binding domains to the RNAP to assemble simple inter- and intra-molecular DNA devices. In one set of experiments, a portion of the yeast GAL4 binding protein was fused to T7 RNAP in a manner consistent with and employing materials and methodology disclosed in Ostrander et al., Science 249: 1261-65 (1990). This modification allows the fusion protein to bind to a DNA fragment that contains the 17 bp GAL4 recognition sequence. In another experiment, we fused the sequence specific zinc-finger DNA binding domain found in the murine transcription factor Zif268 to T7 RNAP. Such domains have been fused to a number of other proteins to confer novel binding capacities on the fusion protein. For the detailed methodology, see the disclosures in Choo, & Isalan, Cur. Op. Struct. Biol. 10: 411-16 (2000); Kim & Paba 1998, supra; Liu et al., Proc. Nat. Acad. Sci. 94: 5525-30 (1997); Smith et al., Nuc. Acids Res. 27: 674-81 (1999). Through genetic manipulations, a broad collection of three finger peptides that each bind to a specific recognition sequence have been developed (Ibid., supra) and may be used and tested for cargo carrying capacity in a fashion analogous to the SBP-tagged RNAP constructed and tested for cargo carrying capacity above.

### EXAMPLE 3

Construction of Simple DNA Devices

To illustrate the ability of a RNAP motor to rearrange a DNA structure, we constructed two simple DNA nanodevices. In the first device, we fused an auxiliary sequence-specific DNA binding domain, the GAL4 binding domain, to T7 RNAP to allow the fusion protein to simultaneously bind to two different DNA regions—the portion of the template DNA being transcribed and the target DNA. Movement of the RNAP along the template changed the disposition of the target DNA relative to the template.

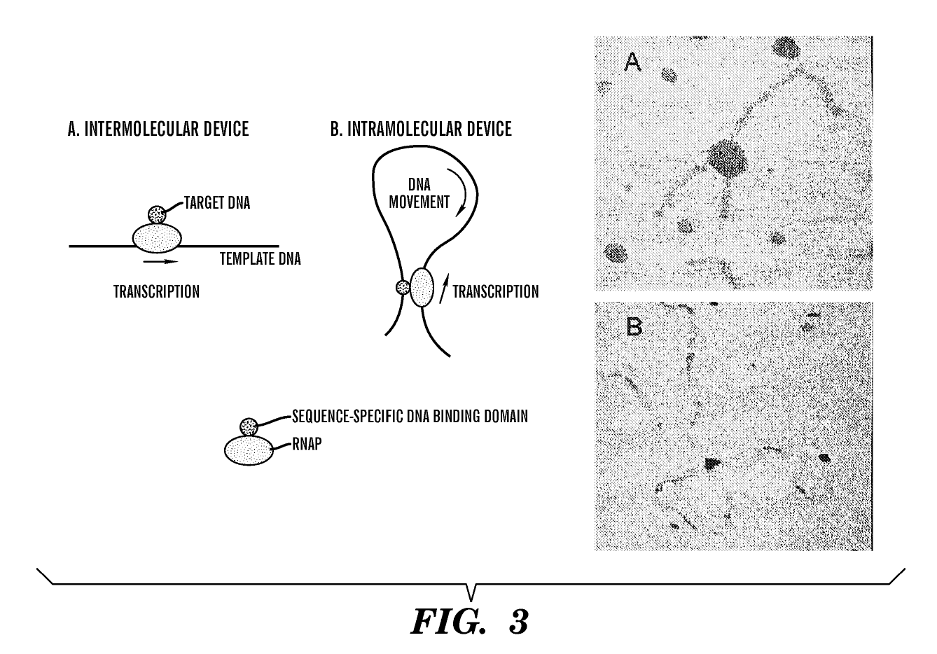

The formation and organization of this type of complex was visualized by atomic force microscopy (AFM) and can be seen in Panel A of FIG. 3. A 1009 bp template DNA containing a T7 promoter 195 bp from one end and a 244 bp target DNA containing a GAL4 binding site near the terminus were prepared by PCR amplification of appropriate plasmids using standard techniques known in the art. The two DNA fragments were incubated with GAL4:T7 RNAP in the presence of G, A and U, which allowed transcription to proceed 22 nt downstream from the promoter in the template DNA. The samples were fixed with formaldehyde and visualized by AFM (tapping mode). The result of the AFM is shown in Panel A, Fig.3. For comparison purposes, a linearlized plasmid containing a T7 promoter and the GAL4 binding site separated by 1 kb were treated in the same manner and visualized by AFM. The result is shown in Panel B, FIG. 3.

As illustrated in FIG. 3, the target sequence may be in a second DNA molecule (A) or in the same molecule (B). When the targeted sequence is in the same molecule, transcription results in the formation of a loop whose dimensions may be increased or decreased, depending upon the direction of transcription. To assemble this device, the target sequence and the transcribed sequence are placed in the same DNA molecule; simultaneous binding of the fusion protein to the target sequence and to the transcribed region results in looping out of the intervening portion. Movement of the RNAP either enlarges or diminished the size of the loop, depending upon the orientation of the promoter in the transcribed region. See FIG. 3, B.

### EXAMPLE 4

Force Measurement of the RNAP Motor under Load

Earlier studies with E. coli RNAP showed that as the enzyme moves along the DNA it can exert a linear force up to 30 pN (stall force) and a rotary force of 5 pN-nm. We anticipate that single subunit RNAPs will exert similar or larger forces. To measure these forces, the methods and approaches developed for the multi-subunit RNAPs were employed. These methods and approaches are disclosed in detail in Davenport 2001, supra; Wang 1998, supra; Yin 1995, supra.

In Example 2 we used gel electrophoresis to demonstrate the ability of T7 RNAP to capture a ligand, move along the template, and release the bound ligand. While these experiments demonstrated controlled movement of the RNAP:ligand complex, they did not provide a direct visualization of the process, nor did they allow measurement of the forces involved. These issues are addressed by the approach described below, in which we demonstrate the use of T7 RNAP as a rotary motor.

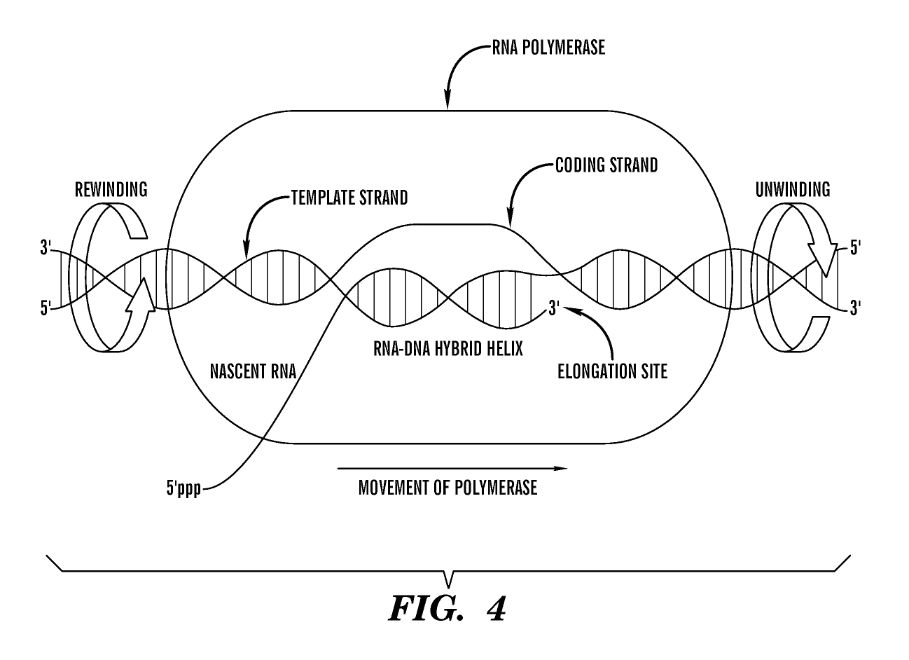

When the polymerase advances along the DNA during transcription, it must unwind the DNA helix at the leading (downstream) edge of the transcription complex and rewind the DNA at the trailing edge, resulting in rotation of the helix relative to the RNAP. To accomplish this, RNAP establishes a locally denatured transcription bubble that encloses an RNA:DNA hybrid of ˜8 bp. As the RNAP advances along the DNA it must unwind the two DNA strands at the leading edge of the bubble and reanneal the strands at the trailing edge. See FIG. 4.

Each step of nucleotide incorporation corresponds to 0.34 nm of linear translocation and 36° of rotation, and depends upon the availability of the next incoming substrate nucleotide as directed by the sequence of the DNA template. Movement of the RNAP is controlled in an information-dependent manner by withholding or adding appropriate substrates.

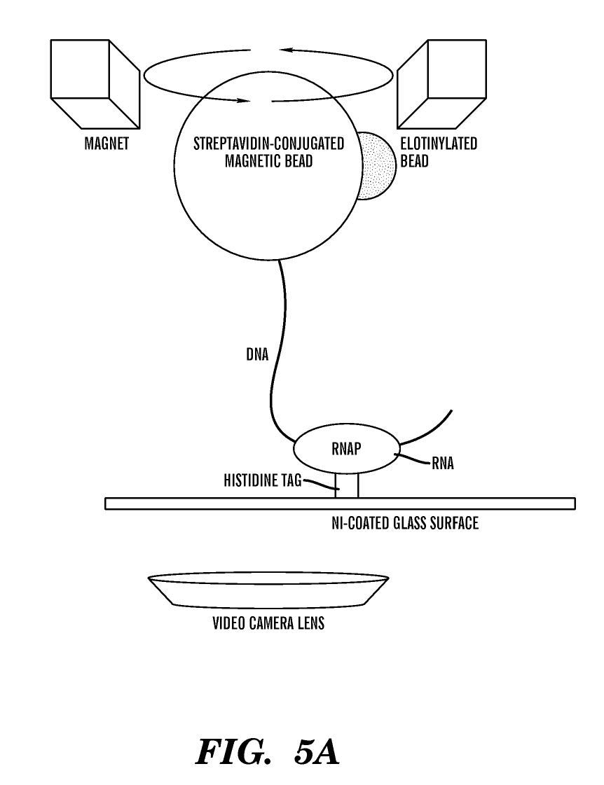

To explore the potential of T7 RNAP as a rotary motor, an elongation complex of T7 RNAP was immobilized on a solid surface using a modified form of T7 RNAP (histidine-tagged T7 RNAP) that has a high affinity for Ni⁺⁺, and the downstream end of a 4 kb DNA template was tethered to a magnetic bead by means of a biotin-streptavidin linkage. The downstream end of the template ws tethered to a streptavidin-conjugated 2.8 μm magnetic bead attached to a smaller, 1.0 μm bead. The complexes were injected between two sealed cover glass slides and immobilized on the bottom slide, which was pre-coated with Ni⁺⁺-NTA. These procedures are illustrated in FIG. 5A.

The assembly was placed in a magnetic tweezers device that allowed the bead to be positioned above the surface and to be visualized by microscopy. After visualizing a single magnetic bead trapped by magnetic tweezers, further transcription was initiated by the addition of substrates, and rotation of the bead was visualized and recorded at 20 frames per second. The data shown in FIG. 5B corresponds to an interval of 2.4 seconds. During transcript elongation the helical template rotates as it passes through the immobilized RNAP, and this force is transmitted to the magnetic bead via the downstream DNA. Under the conditions used in this experiment, the bead was observed to rotate steadily at a rate of ˜30 rpm. Based upon the size of the bead and the viscosity of the solution, we estimate that the RNAP exerts a rotary force of ˜60 pN. nm, which is within the range reported for F₁ATPase. Templates can readily be designed that allow for control of the rotation of the bead in incremental steps according to the sequence of the DNA. The behavior of the RNAP motor can be determined over a wide range of applied tensions to examine how the motor behaves during active transcription, particularly during walking in one nt intervals, or when halted. This is accomplished by repeating the initial experiments but with the modification that one or more NTPs are withheld as in Examples 1-2 above. The actual behavior of the RNAP motor at different stages during the transcription process can then be assessed.

### EXAMPLE 5

Assembly of Arrays of RNAP Motors

In Example 3 above, we described the construction of simple DNA nanodevices involving inter- or intramolecular interactions between an RNAP fusion protein and a target DNA sequence. In this example, we describe the assembly of arrays of RNAP motors on DNA templates, i.e., “bridges” that have been immobilized on a solid surface. Each motor may have a distinct ligand specificity, could be addressed to a specific location on the bridge, and could be independently moved along the template in a sequence-dependent manner.

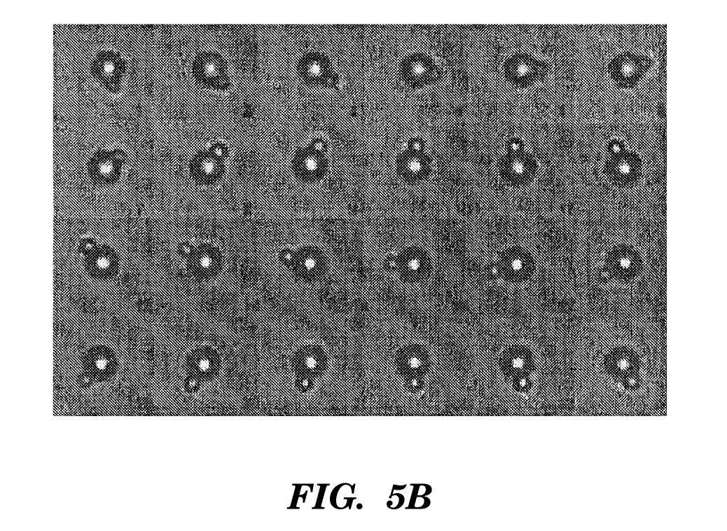

For many applications, it is necessary to fix the DNA template on a solid surface so that the trajectory of the RNAP may be controlled. Two techniques can be used to do this. In the first, DNA bridges are assembled between two locations on a surface by annealing single-stranded regions at the ends of the DNA to complementary oligomers that were previously deposited on the surface. The materials and methods disclosed in Braun et al., Nature 391: 775-78 (1998) may be employed for this purpose. As illustrated in FIG. 6, DNA bridges are assembled between two locations on a surface by annealing single stranded regions at the ends of the DNA to previously deposited oligonucleotides (“anchor oligos”). Separate oligomers for each end of the DNA are used to ensure that all templates in the bridge share the same orientation. Phage promoters are included in the bridge DNA so that the RNAP motors can be directed to bind to the bridge at specific locations and with a particular orientation. The availability of phage RNAPs with unique promoter specificities, for example, T7, T3, SP6 and K11, allows each RNAP motor to be placed at a particular location on the bridge. By fusion to different binding domains, each RNAP motor may be engineered to bind to a specific ligand. The DNA population in the bridge can be homogeneous (homogeneous parallel arrays) or heterogeneous (heterogeneous parallel arrays). Many separate bridges can be deposited on the same surface, in adjacent arrays.

Alternatively, DNA can be immobilized via interactions with hydrophobic patches or strips deposited on a glass surface. Using a controlled combing technique to stretch the DNA during attachment, it was determined that over stretched DNA was not suitable for transcription but that DNA deposited at near contour length was actively transcribed by T7 RNAP. The materials and methods disclosed in Gueroui et al, Proc. Nat. Acad. Sci. 99: 6005-10 (2002); Gueroui et al., EPJ in press (2003) are employed in this technique. Transcription is visualized by incorporation of fluorescently labeled ribonucleotide substrates into individual complexes. Fluorescence microscopy is conveniently used in a flow cell, which is essential for exchanging and delivering substrate during the sequential steps. (The technique can resolve excursions of about 100 bp in complexes that are separated by at least 1 kb.) Consequently, RNAP tagged with the SBP domain as detailed in Examples 1-3 above is used and the RNAP is labeled by binding to streptavidin-conjugated fluorescent beads or nanodots.

In prior work, the assembly of bridge molecules used DNA templates of at least ˜10 kb in length, e.g., the 40 kb bacteriophage T7 genome, which contains 17 phage promoters all oriented in the same direction. See Gueroui et al., EPJin press (2003), supra. Alternatively, templates may be constructed having only 1 or 2 phage promoters, positioned upstream from cassettes that allow controlled movement in increments of 50-100 bp. The promoters are separated by 1 kb and are arranged in either tandem or opposing orientations. By these means the density of loading of the RNAP motors and their relative motions in either orientation may be explored.

Templates are constructed using standard recombinant methods and employing bacteriophage lambda or other well known plasmids or vectors. To construct cassettes suitable for walking in 50-100 bp increments, DNA modules that direct the synthesis of C-less runs of RNA having a composition of (GAU)₅₀ are synthesized. The modules are ligated in tandem using linkers that contain unique runs of . . . CCC. . . . By alternating cycles in which either C or G, A, and U are provided as substrate(s), the polymerase will be limited to a 50 bp walk, or excursion, during each cycle. To prevent recombination between the GAU repeats during construction and propagation of the template, the internal sequence of the GAU module is randomized during synthesis.

### EXAMPLE 6

Construction of Two Dimensional DNA Grids

In some circumstances it may be desirable to construct more complex DNA platforms upon which RNAP motors can be placed and moved. A variety of DNA structures constructed by self-assembly of complementary oligonucleotides are already known in the art and could be adaptable for this purpose. See Chen et al., Nature 350: 631-33 (1991); Seeman, Trends Biochem. Sci. 77:437-42 (1999) for materials and methods to construct such DNA platforms. Oligomers of DNA have been deposited in regular patterns with spacing of 100 nm; the oligomers retain their sequence-specific binding properties. Demers et al., Science 296: 1836-38 (2002). Such patches of oligomers may be used to anchor DNA molecules with complementary ends, allowing the directed assembly of DNA grids. Studies have shown that immobilized DNA molecules may be used as a scaffold on which to deposit or assemble secondary substances such as colloidal gold or other compounds with desirable electrical or mechanical properties. Braun 1998, supra; Alivsatos, Nature 382: 609-11 (1996); Mbindyo et al., Adv. Mater. 13: 249-54 (2001); Mirkin et al., Nature: 607-09 (1996).

Alternatively, connecting molecules could be engineered to allow the assembly of two-dimensional DNA grids in a sequence specific manner. For example, three-finger zinc proteins of the Cys₂-His₂ type may be engineered to bind to a wide variety of DNA sequences with high affinities (K_{d}˜10⁻¹² M) following the methods and employing the materials disclosed in Choo and Isalan supra, Kim, supra, Liu supra, and Smith supra. Three-finger domains can be fused to each other, resulting in six finger peptides that bind to an 18 bp recognition sequence with even higher affinities (K_{d}˜10⁻¹⁵ M) following the methods and employing the materials disclosed in Kim, supra.

By fusing two of these peptides together, peptides having divalent binding capacities can be manufactured. To bind the two binding domains together, a linker peptide should be employed. For recognition of two adjacent 9 bp sequences in the same DNA molecule, a flexible linker (spacer or connector) of ˜12 amino acids is required. Kim, supra. This linker is used to construct a bivalent “connector” molecule having two three-finger domains, one with specificity for the 9 bp Zif268 DNA sequence and the other with specificity for the 9 bp sequence recognized by transcription factor Sp1.

Binding of the fusion protein to these target sequences, either separately when only one target DNA is present, or together when both target molecules are present, is then determined by means of gel-shift assays known in the art. To allow greater spacing and flexibility between the two binding domains, the length of the peptide linker may be increased or an intervening protein domain may be inserted.

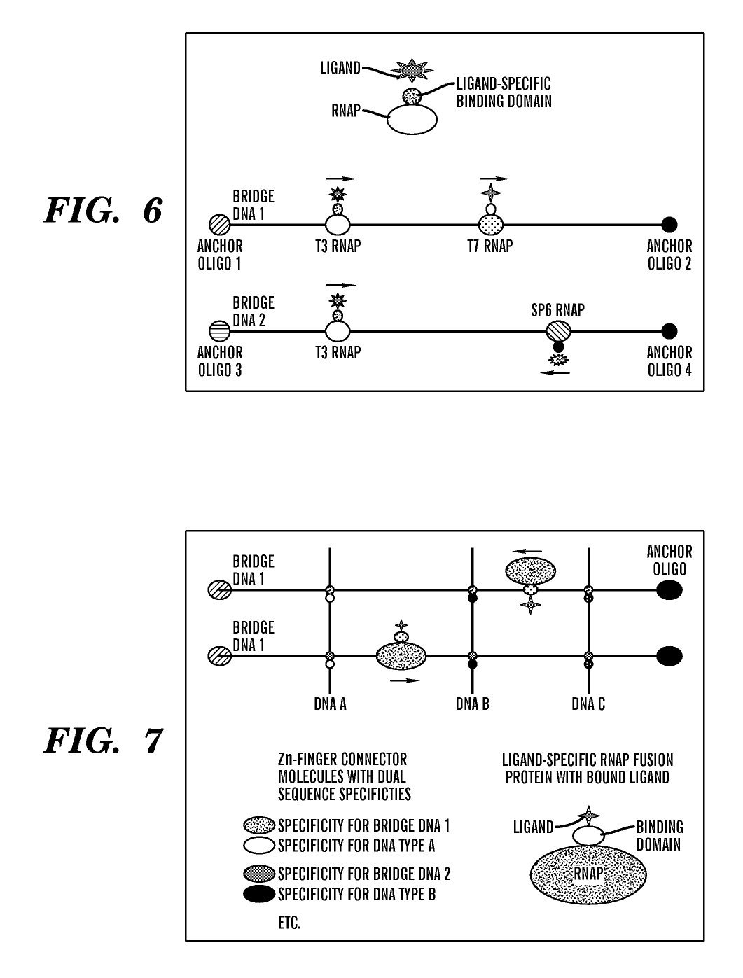

Such a two dimensional grid is illustrated in FIG. 7 wherein connector molecules that contain dual zinc finger binding domains, each with a separate sequence-specific binding capacity, are used to link target sequences engineered into immobilized bridge DNA molecules (horizontal lines) and cross grid molecules (vertical lines). Promoters for RNAP motors may be engineered into the bridge and cross grid DNA molecules, allowing the placement and controlled movement of the motors within the grid. Because T7 RNAP transcription complexes displace bound proteins such as the lac repressor with great efficiency, we anticipate that T7 RNAP will be able to displace divalent three-finger connector molecules such as those described above. See Giordano et al, Gene 84:209-19 (1989). To confirm this, standard transcription assays well known in the art are employed on templates that contain such binding sites in the presence and absence of the connector proteins and/or in the presence of the junction (non-template) DNA. Six-finger zinc finger proteins with higher DNA affinities may be employed in the same manner.

1. A molecular motor for actuating cargo in a controlled and information-dependent manner comprising a nucleotide polymerase enzyme (NP) having a high-affinity binding domain capable of binding to the cargo and being able to move along a DNA template.

2. The motor according to claim 1 wherein the high-affinity binding domain is reversibly or irreversibly bound by attachment or fusion to the NP.

3. The motor according to claim 1 wherein the NP is a single-subunit NP.

4. The motor according to claim 3 wherein the high-affinity binding domain is bound at or near the N-terminus of the NP.

5. The motor according to claim 4 wherein the binding domain is capable of binding to the cargo and to a solid surface.

6. The motor according to claim 4 wherein the high-affinity binding domain is an amino acid sequence.

7. The motor according to claim 6 wherein the amino acid sequence is selected from a yeast GAL4 polypeptide sequence, a Zif268 zinc-finger polypeptide sequence, a streptavidin polypeptide sequence, a metallotheionein polypeptide sequence, a transcription factor Sp1³⁰ polypeptide sequence, or a histidine sequence.

8. The motor according to claim 4 wherein the high-affinity binding domain is a DNA or RNA sequence.

9. The motor according to claim 8 wherein the DNA or RNA sequence is an aptamer.

10. A linear array of molecular motors comprising of a plurality of nucleotide polymerases as described and claimed in claim 3.

11. The array according to claim 10 wherein each motor is fused to a different high-affinity binding domain.

12. The array according to claim 10 wherein each motor comprises a different, phage NP.

13. A plurality of linear arrays according to claim 12, arranged and positioned in a two dimensional grid.

14. A method of actuating cargo in an information dependent manner comprising the steps of (a) creating a start-up complex by adding to a solution of a DNA template having a RNA polymerase promoter and the molecular motor according to claim 1 and sufficient nucleotide triphosphates complementary to the nucleic acids of the DNA template to cause formation of a stable EC, (b) washing the solution to remove excess substrate, (c) adding substrate to the solution, the substrate being composed of one or more nucleotide triphosphates complementary to the nucleic acid(s) of the DNA template downstream from the location of the stable EC, (c) incubating the solution under suitable transcription conditions.

15. The method of actuating cargo according to claim 14 wherein the start-up complex is formed by the addition of at least 14 nucleotide triphosphates complementary to the DNA template downstream from the polymerase binding site.

16. The method of actuating cargo according to claim 15 further comprising the steps of adding in a sequential manner a substrate composed of nucleotide triphosphate complementary to each successive nucleic acid in the DNA template sequence and washing the solution after each addition to remove excess substrate.

17. A method of making a molecular motor comprising binding a NP enzyme to a high affinity binding domain capable of binding to a surface support or a ligand sequence.

18. The method of claim 17 wherein the NP enzyme is a single subunit NP enzyme.

19. The method of claim 18 wherein the high affinity binding domain is a DNA sequence, a RNA sequence or an amino acid sequence selected from a yeast GAL4 polypeptide sequence, a Zif268 zinc-finger polypeptide sequence, a streptavidin polypeptide sequence, a metallotheionein polypeptide sequence, a transcription factor Sp1³⁰ polypeptide sequence, a β-galactosidase polypeptide sequence or a histidine sequence.

20. The method of claim 19 wherein the high affinity binding domain is fused to the NP at or near its N-terminus.
